SEQUENCE LISTING (86 sequences)

SEQ ID NO: 1 - DNA, 27 bp, Artificial Sequence
  Synthetic oligonucleotide
agaggtcatagaaggttatgaaatagc

SEQ ID NO: 2 - DNA, 26 bp, Artificial Sequence
  Synthetic oligonucleotide
gaggtacgaagtagagatgagacttc

SEQ ID NO: 3 - DNA, 23 bp, Artificial Sequence
  Synthetic oligonucleotide
cagcaaggttgaaattgggaatg

SEQ ID NO: 4 - DNA, 26 bp, Artificial Sequence
  Synthetic oligonucleotide
gagtaggagagtggttgaggaaatcc

SEQ ID NO: 5 - DNA, 28 bp, Artificial Sequence
  Synthetic oligonucleotide
caaactggaatagctagcatgtgcttgc

SEQ ID NO: 6 - DNA, 26 bp, Artificial Sequence
  Synthetic oligonucleotide
ggacattcccaatttcaaccttgctg

SEQ ID NO: 7 - DNA, 26 bp, Artificial Sequence
  Synthetic oligonucleotide
gggacatgatttgtaaagttcaaggc

SEQ ID NO: 8 - DNA, 26 bp, Artificial Sequence
  Synthetic oligonucleotide
cacattctgtgaccaaacggttcaac

SEQ ID NO: 9 - DNA, 25 bp, Artificial Sequence
  Synthetic oligonucleotide
ccacagggctaaagcaaccatctcc

SEQ ID NO: 10 - DNA, 25 bp, Artificial Sequence
  Synthetic oligonucleotide
ctcccttcttatgacccaagtggct

SEQ ID NO: 11 - DNA, 24 bp, Artificial Sequence
  Synthetic oligonucleotide
cagggaaaatgaccttcactgctg

SEQ ID NO: 12 - DNA, 26 bp, Artificial Sequence
  Synthetic oligonucleotide
catcccctttaccttagtttacccac

SEQ ID NO: 13 - DNA, 23 bp, Artificial Sequence
  Synthetic oligonucleotide
gtgctggtggcagtgttatttcc

SEQ ID NO: 14 - DNA, 26 bp, Artificial Sequence
  Synthetic oligonucleotide
agtaatgtgttgtcagttcactgagg

SEQ ID NO: 15 - DNA, 19 bp, Artificial Sequence
  Synthetic oligonucleotide
ggagctgcgacacggagaa

SEQ ID NO: 16 - DNA, 19 bp, Artificial Sequence
  Synthetic oligonucleotide
caagcacacctgctgttca

SEQ ID NO: 17 - DNA, 22 bp, Artificial Sequence
  Synthetic oligonucleotide
cagtgcgcgaaatgtagttttg

SEQ ID NO: 18 - DNA, 25 bp, Artificial Sequence
  Synthetic oligonucleotide
gtgtagcacaaaccacagaggagac

SEQ ID NO: 19 - DNA, 24 bp, Artificial Sequence
  Synthetic oligonucleotide
cctcctccctgtcttctctgattc

SEQ ID NO: 20 - DNA, 23 bp, Artificial Sequence
  Synthetic oligonucleotide
caaacagaggtgtgcagcagagg

SEQ ID NO: 21 - DNA, 27 bp, Artificial Sequence
  Synthetic oligonucleotide
ctgccttttgaaccagttagtctggag

SEQ ID NO: 22 - DNA, 25 bp, Artificial Sequence
  Synthetic oligonucleotide
tcccttctctactctgactcctacc

SEQ ID NO: 23 - DNA, 24 bp, Artificial Sequence
  Synthetic oligonucleotide
cctcaacaggagagcagaaggctc

SEQ ID NO: 24 - DNA, 24 bp, Artificial Sequence
  Synthetic oligonucleotide
gtgtgcttcaaggctcagttagtg

SEQ ID NO: 25 - DNA, 20 bp, Artificial Sequence
  Synthetic oligonucleotide
ccccatgctgcccagtcctg

SEQ ID NO: 26 - DNA, 27 bp, Artificial Sequence
  Synthetic oligonucleotide
ctaggttctctacggcctcttgttact

SEQ ID NO: 27 - DNA, 24 bp, Artificial Sequence
  Synthetic oligonucleotide
gcgttcttgttctctagcttcctg

SEQ ID NO: 28 - DNA, 24 bp, Artificial Sequence
  Synthetic oligonucleotide
gataccgatgtcagaggcaggagg

SEQ ID NO: 29 - DNA, 26 bp, Artificial Sequence
  Synthetic oligonucleotide
gttttgtttctcttctctgctgtcgg

SEQ ID NO: 30 - DNA, 26 bp, Artificial Sequence
  Synthetic oligonucleotide
ccaggtggaatctgaatcaagtgtac

SEQ ID NO: 31 - DNA, 18 bp, Artificial Sequence
  Synthetic oligo peptide nucleic acid
  modified_base (1)..(18): peptide nucleic acid (PNA)
atttggtatgttgttctg

SEQ ID NO: 32 - DNA, 16 bp, Artificial Sequence
  Synthetic oligo peptide nucleic acid
  modified_base (1)..(16): peptide nucleic acid (PNA)
tcatcccccaacacaa

SEQ ID NO: 33 - DNA, 18 bp, Artificial Sequence
  Synthetic oligo peptide nucleic acid
  modified_base (1)..(18): peptide nucleic acid (PNA)
gttcttaatagcaggtac

SEQ ID NO: 34 - DNA, 18 bp, Artificial Sequence
  Synthetic oligo peptide nucleic acid
  modified_base (1)..(18): peptide nucleic acid (PNA)
gactcttattggatacag

SEQ ID NO: 35 - DNA, 16 bp, Artificial Sequence
  Synthetic oligo peptide nucleic acid
  modified_base (1)..(16): peptide nucleic acid (PNA)
ggtatgttgtgtgatg

SEQ ID NO: 36 - DNA, 17 bp, Artificial Sequence
  Synthetic oligo peptide nucleic acid
  modified_base (1)..(17): peptide nucleic acid (PNA)
ggtatggttcccttaga

SEQ ID NO: 37 - DNA, 15 bp, Artificial Sequence
  Synthetic oligo peptide nucleic acid
  modified_base (1)..(15): peptide nucleic acid (PNA)
cccagtcgtcagcaa

SEQ ID NO: 38 - DNA, 16 bp, Artificial Sequence
  Synthetic oligo peptide nucleic acid
  modified_base (1)..(16): peptide nucleic acid (PNA)
ctcaccaaactcccag

SEQ ID NO: 39 - DNA, 14 bp, Artificial Sequence
  Synthetic oligo peptide nucleic acid
  modified_base (1)..(14): peptide nucleic acid (PNA)
gaaccccgctaagg

SEQ ID NO: 40 - DNA, 14 bp, Artificial Sequence
  Synthetic oligo peptide nucleic acid
  modified_base (1)..(14): peptide nucleic acid (PNA)
gggcttgttcagct

SEQ ID NO: 41 - DNA, 15 bp, Artificial Sequence
  Synthetic oligo peptide nucleic acid
  modified_base (1)..(15): peptide nucleic acid (PNA)
ttctgggtcaagcct

SEQ ID NO: 42 - DNA, 15 bp, Artificial Sequence
  Synthetic oligo peptide nucleic acid
  modified_base (1)..(15): peptide nucleic acid (PNA)
agctccatagcagtg

SEQ ID NO: 43 - DNA, 16 bp, Artificial Sequence
  Synthetic oligo peptide nucleic acid
  modified_base (1)..(16): peptide nucleic acid (PNA)
tggtcctcatctgctg

SEQ ID NO: 44 - DNA, 15 bp, Artificial Sequence
  Synthetic oligo peptide nucleic acid
  modified_base (1)..(15): peptide nucleic acid (PNA)
gctcgaatttcagag

SEQ ID NO: 45 - DNA, 18 bp, Artificial Sequence
  Synthetic oligo peptide nucleic acid
  modified_base (1)..(18): peptide nucleic acid (PNA)
cactggcttatcatgtct

SEQ ID NO: 46 - DNA, 18 bp, Artificial Sequence
  Synthetic oligo peptide nucleic acid
  modified_base (1)..(18): peptide nucleic acid (PNA)
attattccgaactctagc

SEQ ID NO: 47 - DNA, 16 bp, Artificial Sequence
  Synthetic oligo peptide nucleic acid
  modified_base (1)..(16): peptide nucleic acid (PNA)
cagacctaagttcaag

SEQ ID NO: 48 - DNA, 16 bp, Artificial Sequence
  Synthetic oligo peptide nucleic acid
  modified_base (1)..(16): peptide nucleic acid (PNA)
gatgattctgagcaca

SEQ ID NO: 49 - DNA, 15 bp, Artificial Sequence
  Synthetic oligo peptide nucleic acid (PNA)
  modified_base (1)..(15): peptide nucleic acid (PNA)
ccccaggctgcttat

SEQ ID NO: 50 - DNA, 15 bp, Artificial Sequence
  Synthetic oligo peptide nucleic acid
  modified_base (1)..(15): peptide nucleic acid (PNA)
tgactctaaagcaga

SEQ ID NO: 51 - DNA, 18 bp, Artificial Sequence
  Synthetic oligo peptide nucleic acid
  modified_base (1)..(18): peptide nucleic acid (PNA)
ctctagttcgccatagcc

SEQ ID NO: 52 - DNA, 17 bp, Artificial Sequence
  Synthetic oligo peptide nucleic acid
  modified_base (1)..(17): peptide nucleic acid (PNA)
ccaccattagtgcctct

SEQ ID NO: 53 - DNA, 15 bp, Artificial Sequence
  Synthetic oligo peptide nucleic acid
  modified_base (1)..(15): peptide nucleic acid (PNA)
cctcaagccacacaa

SEQ ID NO: 54 - DNA, 18 bp, Artificial Sequence
  Synthetic oligo peptide nucleic acid
  modified_base (1)..(18): peptide nucleic acid (PNA)
tgtcctcagcctttctcg

SEQ ID NO: 55 - DNA, 17 bp, Artificial Sequence
  Synthetic oligo peptide nucleic acid
  modified_base (1)..(17): peptide nucleic acid (PNA)
cccttcactgtcatcct

SEQ ID NO: 56 - DNA, 15 bp, Artificial Sequence
  Synthetic oligo peptide nucleic acid
  modified_base (1)..(15): peptide nucleic acid (PNA)
ccagcagcctccaca

SEQ ID NO: 57 - DNA, 15 bp, Artificial Sequence
  Synthetic oligo peptide nucleic acid
  modified_base (1)..(15): peptide nucleic acid (PNA)
gctgtgtcagtcctg

SEQ ID NO: 58 - DNA, 18 bp, Artificial Sequence
  Synthetic oligo peptide nucleic acid
  modified_base (1)..(18): peptide nucleic acid (PNA)
cagttgacattagtaaat

SEQ ID NO: 59 - DNA, 16 bp, Artificial Sequence
  Synthetic oligo peptide nucleic acid
  modified_base (1)..(16): peptide nucleic acid (PNA)
ctcccgagctgactcc

SEQ ID NO: 60 - DNA, 14 bp, Artificial Sequence
  Synthetic oligo peptide nucleic acid
  modified_base (1)..(14): peptide nucleic acid (PNA)
aaatccgccctgac

SEQ ID NO: 61 - DNA, 13 bp, Artificial Sequence
  Synthetic oligonucleotide
gatacagtgcagc

SEQ ID NO: 62 - DNA, 14 bp, Artificial Sequence
  Synthetic oligonucleotide
gatacagtgcagcg

SEQ ID NO: 63 - DNA, 15 bp, Artificial Sequence
  Synthetic oligonucleotide
ggatacagtgcagcg

SEQ ID NO: 64 - DNA, 14 bp, Artificial Sequence
  Synthetic oligonucleotide
gtgtgatgatcagc

SEQ ID NO: 65 - DNA, 15 bp, Artificial Sequence
  Synthetic oligonucleotide
gtgtgatgatcagca

SEQ ID NO: 66 - DNA, 16 bp, Artificial Sequence
  Synthetic oligonucleotide
gtgtgatgatcagcac

SEQ ID NO: 67 - DNA, 18 bp, Artificial Sequence
  Synthetic oligonucleotide
atttggtatgttgttctg

SEQ ID NO: 68 - DNA, 16 bp, Artificial Sequence
  Synthetic oligonucleotide
tcatcccccaacacaa

SEQ ID NO: 69 - DNA, 18 bp, Artificial Sequence
  Synthetic oligonucleotide
gttcttaatagcaggtac

SEQ ID NO: 70 - DNA, 18 bp, Artificial Sequence
  Synthetic oligonucleotide
gactcttattggatacag

SEQ ID NO: 71 - DNA, 14 bp, Artificial Sequence
  Synthetic oligonucleotide
ggtatgaggtgtga

SEQ ID NO: 72 - DNA, 18 bp, Artificial Sequence
  Synthetic oligonucleotide
atttggtacgttgttctg

SEQ ID NO: 73 - DNA, 16 bp, Artificial Sequence
  Synthetic oligonucleotide
tcatctcccagcacaa

SEQ ID NO: 74 - DNA, 18 bp, Artificial Sequence
  Synthetic oligonucleotide
gttcttaacagcaggtac

SEQ ID NO: 75 - DNA, 18 bp, Artificial Sequence
  Synthetic oligonucleotide
gactcttactggatacag

SEQ ID NO: 76 - DNA, 14 bp, Artificial Sequence
  Synthetic oligonucleotide
ggtatgaggtgtga

SEQ ID NO: 77 - DNA, 15 bp, Artificial Sequence
  Synthetic oligonucleotide
ttctggatcaagcct

SEQ ID NO: 78 - DNA, 14 bp, Artificial Sequence
  Synthetic oligonucleotide
agctccgtagcagt

SEQ ID NO: 79 - DNA, 15 bp, Artificial Sequence
  Synthetic oligonucleotide
ggtcctcgtctgctg

SEQ ID NO: 80 - DNA, 15 bp, Artificial Sequence
  Synthetic oligonucleotide
gctcgagtttcagag

SEQ ID NO: 81 - DNA, 18 bp, Artificial Sequence
  Synthetic oligonucleotide
cactggctcatcatgtct

SEQ ID NO: 82 - DNA, 18 bp, Artificial Sequence
  Synthetic oligonucleotide
ctctagttctccatagcc

SEQ ID NO: 83 - DNA, 17 bp, Artificial Sequence
  Synthetic oligonucleotide
ccaccatcagtgcctct

SEQ ID NO: 84 - DNA, 15 bp, Artificial Sequence
  Synthetic oligonucleotide
cctcaaaccacacaa

SEQ ID NO: 85 - DNA, 18 bp, Artificial Sequence
  Synthetic oligonucleotide
tgtcctcaacctttctcg

SEQ ID NO: 86 - DNA, 17 bp, Artificial Sequence
  Synthetic oligonucleotide
cccttcattgtcatcct

(19) United States
(12) Patent Application Publication
(10) Pub. No.: US 2022/0127665 A1
(43) Pub. Date: Apr. 28, 2022

(54) ELIMINATION PROBE-BASED METHOD FOR DETECTING NUMERICAL CHROMOSOMAL ABNORMALITIES, AND NUCLEIC ACID COMPOSITION FOR DETECTING NUMERICAL CHROMOSOMAL ABNORMALITIES

(71) Applicant: SEASUN BIOMATERIALS, Daejeon (KR)

(72) Inventors: Si Seok LEE, Daejeon (KR); Kyung Tak KIM, Daejeon (KR); Eun Ju YANG, Daejeon (KR); Hee Kyung PARK, Daejeon (KR)

(21) Appl. No.: 17/265,102

(22) PCT Filed: Jul. 23, 2019
(86) PCT No.: PCT/KR2019/009067

§ 371 Date: Feb. 1, 2021

(30) Foreign Application Priority Data
Jul. 31, 2018 (KR) 10-2018-0089224

(51) Int. Cl. C12Q 1/6832; C12Q 1/6825; C12Q 1/6818; C12Q 1/686; C12Q 1/6883

(52) U.S. Cl. CPC C12Q 1/6832; C12Q 1/6825; C12Q 2600/156; C12Q 1/686; C12Q 1/6883; C12Q 1/6818

(57) ABSTRACT

The present invention relates to a method for analyzing the presence or absence of aneuploidy of a target chromosome with high sensitivity, and a composition for detecting chromosomal aneuploidy, and more particularly to a method of identifying chromosomal aneuploidy by amplifying a control nucleotide sequence, located on a chromosome not associated with chromosomal aneuploidy, and a target nucleotide sequence located on a chromosome associated with chromosomal aneuploidy, by using the same primer, and then hybridizing the amplification products with an assay probe that differs by one or two nucleotides from the control nucleotide sequence and with an elimination probe that comprises part or all of a sequence of the assay probe, which hybridizes with the target nucleotide sequence or the control nucleotide sequence, the elimination probe having a higher binding affinity for the amplification products than the assay probe, and analyzing melting curves of the hybridization products. The method for detecting chromosomal aneuploidy according to the present invention may analyze the ratio of the target nucleotide sequence to the control nucleotide sequence at high resolution by eliminating equal amounts (certain proportions) of the target nucleotide sequence and the control nucleotide sequence from the analysis using the elimination sequence. This method is useful because numerical abnormalities (aneuploidy) in chromosomes (e.g., fetal chromosomes in maternal blood, and circulating tumor DNA in cancer patients) present at low rates can be detected quickly with high sensitivity by the use of this method.

### SEQUENCE LISTING

The instant application contains a Sequence Listing which has been submitted electronically in ASCII format and is hereby incorporated by reference in its entirety. Said ASCII copy, created Oct. 7, 2021, is named 404266_001 US_SL.txt and is 20,409 bytes in size.

### TECHNICAL FIELD

The present invention relates to a method for analyzing the presence or absence of aneuploidy of a target chromosome with high sensitivity, and a composition for detecting chromosomal aneuploidy, and more particularly to a method of identifying chromosomal aneuploidy by amplifying a control nucleotide sequence, located on a chromosome not associated with chromosomal aneuploidy, and a target nucleotide sequence located on a chromosome associated with chromosomal aneuploidy, by using the same primer, and then hybridizing the amplification products with an assay probe that differs by one or two nucleotides from the control nucleotide sequence or target nucleotide sequence and with an elimination probe that comprises part or all of a sequence of the assay probe, which hybridizes with the target nucleotide sequence or the control nucleotide sequence, the elimination probe having a higher binding affinity for the amplification products than the assay probe, and analyzing melting curves of the hybridization products.

### BACKGROUND ART

Chromosomal abnormalities are associated with genetic defects and degenerative diseases. Chromosome abnormality may indicate deletion or duplication of a chromosome, deletion or duplication of a part of chromosome, or a break, translocation, or inversion in the chromosome. Chromosomal abnormalities are disturbances in the genetic balance and cause fetal death or serious defect in physical and mental states. For example, Down's syndrome is a common form of chromosomal aneuploidy (numerical chromosomal abnormality) caused by the presence of three chromosomes 21 (trisomy 21). Edwards syndrome (trisomy 18), Patau syndrome (trisomy 13), Turner syndrome (XO) and Klinefelter syndrome (XXY) also correspond to chromosomal aneuploidy.

Chromosomal abnormalities may be detected using karyotyping and fluorescent in situ hybridization (FISH). These detection methods are disadvantageous in terms of time, effort and accuracy. Furthermore, karyotyping requires a lot of time for cell culture. FISH is only available for samples of known nucleic acid sequence and chromosomal location. FISH may be used only for samples having known nucleic acid sequences and chromosomal locations. In order to avoid the problems of FISH, comparative genome hybridization (CGH) may be used. CGH may detect a region in which chromosomal aneuploidy has occurred by analyzing the whole genome. However, CGH has a disadvantage in that the resolution thereof is lower than that of FISH.

As an alternative approach, DNA microarrays may be used to detect chromosomal abnormalities. The DNA microarray systems may be classified, according to the type of bio-molecules immobilized on the microarray, into cDNA microarrays, oligonucleotide microarrays, and genomic microarrays. cDNA microarrays and oligonucleotide microarrays are easy to fabricate, but these systems have disadvantages in that the number of probes immobilized on the microarray is limited, probe fabrication is expensive, and it is difficult to detect chromosomal abnormalities located outside the probes.

In particular, in the case of a genomic DNA microarray system, it is easy to fabricate a probe, and it is possible detect chromosomal abnormalities not only in the extended region of the chromosome but also in the intron region of the chromosome, but it is difficult to produce a large number of DNA fragments whose localization and function within the chromosome are known.

Recently, next-generation sequencing technology has been used to analyze numerical chromosomal abnormalities (chromosomal aneuploidy) (Park, H., Kim et al., Nat Genet 2010, 42, 400-405.; Kidd, J. M. et al., Nature 2008, 453, 56-64). However, this technology requires high coverage reading for the analysis of chromosomal aneuploidy, and CNV measurements also require independent validation. Therefore, this technology was not suitable as a general gene search analysis method at that time because it was very expensive and the results were difficult to understand.

For this purpose, real-time qPCR is currently used as an advanced technique for quantitative gene analysis, because a wide dynamic range (Weaver, S. et al, Methods 2010, 50, 271-276) and a linear correlation between the threshold cycle and the initial target amount are observed reproducibly (Deepak, S. et al., Curr Genomics 2007,8, 234-251). However, the sensitivity of qPCR is not high enough to discriminate differences in the number of copies. Despite the wide dynamic range of qPCR assay, small changes, such as 1.5-fold changes, cannot be reliably measured due to the intrinsic variables of qPCR-based assays. In addition, multiple temporally repetitive analyses are required for reliable distinction between samples with similar copies of DNA. Furthermore, qPCR is not suitable for multimodal analysis. For example, for detection of multiple targets, a reaction for separating the targets from each other is required to distinguish one target from others (Bustin, S. A., J Mol Endocrinol 2002, 29, 23-39). In addition, due to the limited availability and spectral overlap of fluorescent tags, qPCR can separate up to only 4 targets per assay. However, for successful quadruplex analysis in qPCR, a careful combination of fluorescent tags is essential for each analysis (Bustin, S A, J Mol Endocrinol 2002, 29, 23-39), which is a serious disadvantage of qPCR as a clinical diagnostic tool.

Accordingly, the present inventors have made extensive efforts to solve the above-described problems and to develop a method for detecting chromosomal aneuploidy, which may provide analysis results quickly with high sensitivity. As a result, the present inventors have found that, when both a control nucleotide sequence and a target nucleotide sequence are amplified and then the amplification product from the control nucleotide sequence is eliminated using an elimination probe, analysis results may be obtained quickly with high sensitivity, thereby completing the present invention.

### SUMMARY OF THE INVENTION

An object of the present invention is to provide a method for detecting chromosomal aneuploidy.

Another object of the present invention is to provide a PCR composition for detecting chromosomal aneuploidy.

To achieve the above objects, the present invention provides a method for detecting chromosomal aneuploidy, the method comprising steps of: a) isolating DNAs from a normal sample and a subject sample, respectively; b) performing amplification using a primer capable of amplifying both a control nucleotide sequence located on a chromosome not associated with chromosomal aneuploidy and a target nucleotide located on a chromosome associated with chromosomal aneuploidy; c) hybridizing the amplification products with an assay probe capable of hybridizing to a sequence, which differs by one or two nucleotides from the control nucleotide sequence or the target nucleotide sequence, and with an elimination probe comprising part or all of a sequence of the assay probe, which hybridizes with the target nucleotide sequence or the control nucleotide sequence, the elimination probe having a higher binding affinity for the amplification products of step b) than the assay probe; and d) identifying chromosomal aneuploidy by analyzing melting curves of the hybridization products for the normal sample and the subject sample, obtained in step c).

The present invention also provides a PCR composition for detecting chromosomal aneuploidy, the PCR composition comprising: i) a primer capable of amplifying both a control nucleotide sequence located on a chromosome not associated with chromosomal aneuploidy and a target nucleotide located on a chromosome associated with chromosomal aneuploidy; ii) an assay probe capable of hybridizing a sequence that differs by one or two nucleotides from the control nucleotide sequence or the target nucleotide sequence; and iii) an elimination probe comprising part or all of a sequence of the assay probe, which hybridizes to the target nucleotide sequence or the control nucleotide sequence, the elimination probe having a higher binding affinity than the assay probe.

The present invention also provides the use of the PCR composition for detection of chromosomal aneuploidy.

### BRIEF DESCRIPTION OF THE DRAWINGS

FIG. 1 is a schematic view showing that normal and abnormal chromosomes are eliminated at the same rate by the use of an elimination probe according to the present invention.

FIG. 2 is a schematic view showing change in analytical resolution depending on the elimination rate by use of the elimination probe according to the present invention.

FIG. 3 is schematic view showing conditions for selecting a target nucleotide sequence according to the present invention and conditions for selecting a primer for amplification of the target nucleotide sequence.

FIG. 4 is a diagram showing real-time PCR conditions for determining whether the chromosome ratio is abnormal according to the present invention.

FIG. 5 is a schematic view showing a detection probe and an elimination probe according to the present invention. FIG. 5(A) shows a non-fluorescent elimination probe that binds to both a target nucleotide sequence and a control nucleotide sequence, FIG. 5(B) shows a non-fluorescent probe that binds only to a control nucleotide sequence, and FIG. 5(C) shows a fluorescent elimination probe that binds only to a control nucleotide sequence.

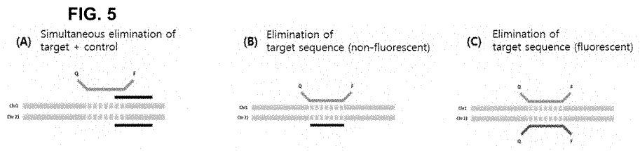

FIG. 6 shows the results of analysis using a Down's syndrome cell line according to the present invention. FIGS. 6(A) and 6(B) show the results of analyses based on different control nucleotide sequences and target nucleotide sequences.

FIG. 7 shows the results of analysis using an Edward's syndrome cell line according to the present invention. FIGS. 7(A) and 7(B) show the results of analyses based on different control nucleotide sequences and target nucleotide sequences.

FIG. 8 shows the results of analysis using a Patau syndrome cell line according to the present invention. FIGS. 8(A) and 8(B) show the results of analyses based on different control nucleotide sequences and target nucleotide sequences.

FIG. 9 illustrates the results of analyzing sensitivity depending on the proportion of DNA using a Down's syndrome cell line according to the present invention. FIGS. 9(A) and 9(B) show the results of analyses based on different control nucleotide sequences and target nucleotide sequences.

FIG. 10 shows results indicating the increase in analytical resolution by the use of a non-fluorescent probe that eliminates only a control nucleotide sequence according to the present invention.

FIG. 11 shows results indicating the increase in analytical resolution by the use of a non-fluorescent probe that simultaneously eliminates a target nucleotide sequence and a control nucleotide sequence according to the present invention.

FIG. 12 is a schematic view showing correction of the results obtained using a fluorescent elimination probe targeting a control nucleotide sequence according to the present invention.

FIG. 13 illustrates the results of correcting the results according to the present invention.

FIG. 14 shows the results of comparatively analyzing a standard substance and a clinical sample.

### DETAILED DESCRIPTION AND PREFERRED EMBODIMENTS OF THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

In the present invention, based on DNA isolated from a normal sample and a subject sample, a target nucleotide sequence located on a chromosome expected to be associated with chromosomal aneuploidy and a control nucleotide sequence located on a chromosome not associated with chromosomal aneuploidy while having at least 90% homology to the target nucleotide sequence were amplified using the same primer, certain amounts of the amplification products were eliminated with an elimination probe, and then melting curves of the amplification products were analyzed using an assay probe. As a result, it was confirmed that chromosomal aneuploidy could be detected with high sensitivity.

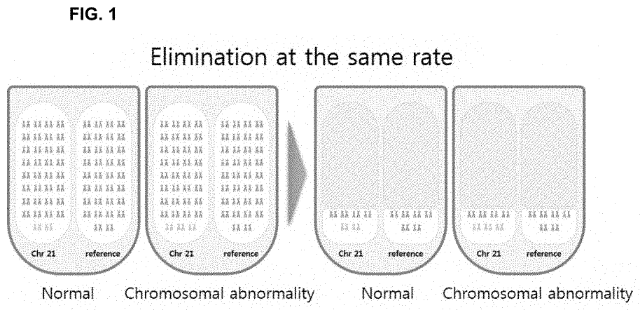

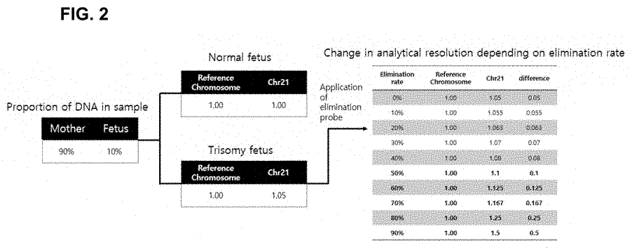

That is, in one example of the present invention, amplification products were produced using a synthesized primer capable of amplifying a certain region of each of chromosomes 1, 4 and 7 while amplifying a certain region of chromosome 21, or a synthesized primer capable of amplifying a certain region of each of chromosomes 1, 4, 9 and 15 while amplifying a certain region of chromosome 19, or a synthesized primer capable of amplifying a certain region of each of chromosomes 3, 6 and 12 while amplifying a certain region of chromosome 13, and then a certain proportion of each of the amplification products was prevented from binding to an assay probe by the use of an elimination probe capable of hybridizing with the amplification products, and then the mismatch/perfect match ratio for the normal sample and the subject sample was calculated by analyzing melting curves. As a result, it was confirmed that chromosomal aneuploidy could be detected with high sensitivity (FIGS. 1 and 2). Therefore, in one aspect, the present invention is directed to a method for detecting chromosomal aneuploidy, the method comprising steps of:
a) isolating DNAs from a normal sample and a subject sample, respectively;
b) performing amplification using a primer capable of amplifying both a control nucleotide sequence located on a chromosome not associated with chromosomal aneuploidy and a target nucleotide located on a chromosome associated with chromosomal aneuploidy;
c) hybridizing the amplification products with an assay probe capable of hybridizing to a sequence, which differs by one or two nucleotides from the control nucleotide sequence or the target nucleotide sequence, and with an elimination probe comprising part or all of a sequence of the assay probe, which hybridizes to the target nucleotide sequence or the control nucleotide sequence, the elimination probe having a higher binding affinity for the amplification products of step b) than the assay probe; and
d) identifying chromosomal aneuploidy by analyzing melting curves of the hybridization products for the normal sample and the subject sample, obtained in step c).

As used herein, the term “target nucleotide sequence” refers to all types of nucleic acids to be detected, and include chromosomal sequences from different species, subspecies or variants, or chromosomal mutations within the same species. The target nucleotide sequence may be characterized by all types of DNA including genomic DNA, mitochondrial DNA, and viral DNA, or all types of RNA including mRNA, ribosomal RNA, non-coding RNA, tRNA, and viral RNA, but is not limited thereto.

In the present invention, the target nucleotide sequence may be a mutant nucleotide sequence including a variation of the nucleotide sequence, and the mutation may be selected from the group consisting of single nucleotide polymorphism (SNP), insertion, deletion, point mutation, fusion mutation, translocation, inversion, and LOH (loss of heterozygosity), but is not limited thereto.

As used herein, the term “nucleoside” refers to a glycosylamine compound wherein a nucleic acid base (nucleobase) is linked to a sugar moiety. The term “nucleotide” refers to a nucleoside phosphate. A nucleotide may be represented using alphabetical letters (letter designation) corresponding to its nucleoside as described in Table 1. For example, A denotes adenosine (a nucleoside containing the nucleobase, adenine), C denotes cytidine, G denotes guanosine, U denotes uridine, and T denotes thymidine (5-methyl uridine). W denotes either A or T/U, and S denotes either G or C. N represents a random nucleoside and dNTP refers to deoxyribonucleoside triphosphate. N may be any of A, C, G, or T/U.

TABLE 1

| Symbol | Nucleotide represented |
|---|---|
| letter | by symbol letter |
| G | G |
| A | A |
| T | T |
| C | C |
| U | U |
| R | G or A |
| Y | T/U or C |
| M | A or C |
| K | G or T/U |
| S | G or C |
| W | A or T/U |
| H | A or C or T/U |
| B | G or T/U or C |
| V | G or C or A |
| D | G or A or T/U |
| H | G or A or T/U or C |

As used herein, the term “oligonucleotide” refers to oligomers of nucleotides. The term “nucleic acid” as used herein refers to polymers of nucleotides. The term “sequence” as used herein refers to a nucleotide sequence of an oligonucleotide or a nucleic acid. Throughout the specification, whenever an oligonucleotide or nucleic acid is represented by a sequence of letters, the nucleotides are in 5′→3′ order from left to right. The oligonucleotides or nucleic acids may be DNA, RNA, or analogues thereof (e.g., phosphorothioate analogue). The oligonucleotides or nucleic acids may also include modified bases and/or backbones (e.g., modified phosphate linkage or modified sugar moiety). Non-limiting examples of synthetic backbones that confer stability and/or other advantages to the nucleic acids may include phosphorothioate linkages, peptide nucleic acid, locked nucleic acid, xylose nucleic acid, or analogues thereof.

As used herein, the term “nucleic acid” refers to a nucleotide polymer, and unless otherwise limited, would encompass known analogs of natural nucleotides that can function in a similar manner (e.g., hybridization) as naturally occurring nucleotides.

The term “nucleic acid” includes, for example, genomic DNA; complementary DNA (cDNA) (which is a DNA representation of mRNA, usually obtained by reverse transcription of messenger RNA (mRNA) or by amplification); DNA molecules produced synthetically or by amplification; and any form of DNA or RNA including mRNA.

The term “nucleic acid” encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands).

The term nucleic acid also encompasses any chemical modification thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, backbone modifications, unusual base pairing combinations such as the isobases isocytidine and isoguanidine, and the like.

The nucleic acid(s) can be derived from a completely chemical synthesis process, such as solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “complementary” refers to refers to the capacity for precise pairing between two nucleotides. That is, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

As used herein, the term “primer” refers to a short linear oligonucleotide that hybridizes with a target nucleic acid sequence (e.g., a DNA template to be amplified) to prime a nucleic acid synthesis reaction. The primer may be an RNA oligonucleotide, a DNA oligonucleotide, or a chimeric sequence. The primer may contain natural, synthetic, or modified nucleotides. Both the upper and lower limits of the length of the primer are empirically determined. The lower limit on the primer length is the minimum length that is required to form a stable duplex upon hybridization with the target nucleic acid under nucleic acid amplification reaction conditions. Very short primers (usually less than 3 to 4 nucleotides long) do not form thermodynamically stable duplexes with target nucleic acid under such hybridization conditions. The upper limit is often determined by the possibility of having duplex formation in a region other than the predetermined nucleic acid sequence in the target nucleic acid. Generally, suitable primer lengths are in the range of about 4 to about 40 nucleotides long.

As used herein, the term “probe” is a nucleic acid capable of binding to a target nucleic acid of complementary sequence through one or more types of chemical bonds, generally through complementary base pairing, usually through hydrogen bond formation, thus forming a duplex structure. The probe binds or hybridizes to a “probe binding site.” The probe can be labeled with a detectable label to permit facile detection of the probe, particularly once the probe has hybridized to its complementary target. Alternatively, however, the probe may be unlabeled, but may be detectable by specific binding with a ligand that is labeled, either directly or indirectly. Probes can vary significantly in size. Generally, probes are at least 7 to 15 nucleotides in length. Other probes are at least 20, 30, or 40 nucleotides long. Still other probes are somewhat longer, being at least 50, 60, 70, 80, or 90 nucleotides long. Yet other probes are longer still, and are at least 100, 150, 200 or more nucleotides long. Probes can also be of any length that is within any range bounded by any of the above values (e.g., 15 to 20 nucleotides in length).

As used herein, the term “hybridization” refers to the formation of a double-stranded nucleic acid by hydrogen bonding between single-stranded nucleic acids having complementary base sequences, and is used in a similar sense to annealing. However, in a broader sense, hybridization includes cases where nucleotides are perfectly complementary (perfect match) between two single-stranded molecules, as well as cases where some nucleotides are not complementary (mismatch).

In the present invention, the amplification is not limited as long as it is polymerase chain reaction (PCR), but it is preferably asymmetric PCR.

In the present invention, the homology of the primer or probe hybridization region of the control nucleotide sequence of step b) is not limited as long as the same probe or primer is capable of binding complementarily to the primer or probe hybridization region of the target nucleotide sequence, but the homology is preferably at least 80%, more preferably at least 90%, most preferably 95%.

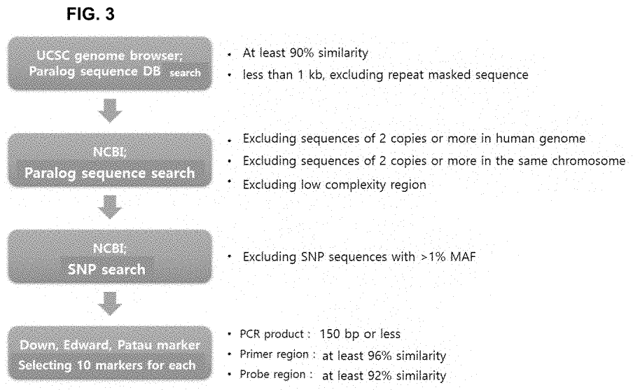

In the present disclosure, the control nucleotide sequence may be selected under the conditions described in FIG. 3.

In the present invention, the assay probe in step c) may be used without limitation as long as the melting temperature difference occurs to the extent that it can be distinguished on the analysis graph, when the assay probe either perfectly matches or mismatches the control nucleotide sequence or the target nucleotide sequence. Preferably, the melting temperature difference may be 5° C. to 20° C., more preferably 7° C. to 20° C., most preferably 8° C. to 20° C.

In the present invention, the assay probe in step c) may be a peptide nucleic acid (PNA), and a reporter and a quencher may be attached to both ends of the assay probe.

In the present invention, the peptide nucleic acid (PNA) is one of substances that recognize genes, like LNA (locked nucleic acid) and MNA (morpholino nucleic acid), and is synthesized artificially, and the backbone thereof is composed of polyamide. PNA has excellent affinity and selectivity, and is not degraded by existing restriction enzymes due to high stability thereof against nucleases. In addition, PNA has an advantage of being easy to store and is not easily degraded due to high thermal/chemical properties and stability thereof. In addition, PNA-DNA binding is much stronger than DNA-DNA binding, and a melting temperature (Tm) difference of about 10 to 15° C. appears even for one nucleotide mismatch. Using this difference in binding strength, it is possible to detect changes in single nucleotide polymorphism (SNP) and insertion/deletion (InDel) nucleic acids.

The Tm value also changes depending on the difference between the nucleotide sequence of the PNA probe and the nucleolide sequence of DNA complementary thereto, and thus the development of applications based on this change is easily achieved. The PNA probe is analyzed using a hybridization reaction different from the hydrolysis reaction of the TaqMan probe, and probes having functions similar to those of the PNA probe include molecular beacon probes and scorpion probes.

In the present invention, a reporter or a quencher may be attached to the PNA probe, without being limited thereto. The PNA probe comprising the reporter and quencher according to the present invention generates a fluorescent signal after hybridization with the target nucleic acid, and as the temperature rises, the fluorescent signal is quenched by rapid melting of the target nucleic acid at an appropriate melting temperature of the probe. Through analysis of a high-resolution melting curve obtained from the fluorescent signal resulting from this temperature change, the presence or absence of the target nucleic acid may be detected.

The probe of the present invention may have a reporter and a quencher capable of quenching reporter fluorescence, attached at both ends thereof, and may include an intercalating fluorophore. The reporter may be one or more selected from the group consisting of FAM (6-carboxyfluorescein), HEX, Texas red, JOE, TAMRA, CY5, CY3, and Alexa680, and the quencher is preferably TAMRA (6-carboxytetramethyl-rhodamine), BHQ1, BHQ2 or Dabcyl, but is not limited thereto. The intercalating fluorophore may be selected from the group consisting of Acridine homodimer and derivatives thereof, Acridine Orange and derivatives thereof, 7-aminoactinomycin D (7-AAD) and derivatives thereof, Actinomycin D and derivatives thereof, 9-amino-6-chloro-2-methoxyacridine (ACMA) and derivatives thereof, DAPI and derivatives thereof, dihydroethidium and derivatives thereof, ethidium bromide and derivatives thereof, ethidium homodimer-1 (EthD-1) and derivatives thereof, ethidium homodimer-2 (EthD-2) and derivatives thereof, ethidium monoazide and derivatives thereof, hexidium iodide and derivatives thereof, bisbenzimide (Hoechst 33258) and derivatives thereof, Hoechst 33342 and derivatives thereof, Hoechst 34580 and derivatives thereof, hydroxystilbamidine and derivatives thereof, LDS 751 and derivatives thereof, propidium iodide (PI) and derivatives thereof, and Cy-dyes derivatives.

In the present invention, the elimination probe in step c) may be selected from the group consisting of: a probe for eliminating only the product of amplification of the target nucleotide sequence; and a probe for eliminating both the products of amplification of the target nucleotide sequence and the control nucleotide sequence.

In the present invention, the elimination probe in step c) may hybridize to the product of amplification of the control nucleotide sequence or the target nucleotide sequence competitively with the assay probe.

In the present invention, the elimination probe may be selected from the group consisting of an oligonucleotide, LNA, PNA, and combinations thereof.

In the present invention, the elimination probe in step c) may eliminate 50 to 90% of the amplification products obtained in step b).

In the present invention, the elimination probe may have a higher Tm value than the assay probe.

In the present invention, analysis of the melting curves in step d) may be performed by a method comprising steps of:
a) calculating the mismatch value/perfect match value ratio of the product of amplification of the normal sample DNA;
b) calculating the mismatch value/perfect match value ratio of the product of amplification of the subject sample DNA; and
c) determining that the subject sample is normal when the ratio calculated in step a) is the same as the ratio calculated in step b), and has chromosomal aneuploidy when the ratio calculated in step a) is different from the ratio calculated in step b).

In the present invention, the analysis of the melting curves may further comprise:
step d) of correcting the perfect match, obtained by the elimination probe value, using the following Equation 1 when calculating the ratios in step a) and step b):

$$Equation 1$$

$$\frac{Mismatch value}{Perfect match value} / \frac{Perfect match value}{Perfect match value by elimination probe}$$

Fluorescence melting curve analysis (FMCA) is used as the method for analyzing the hybridization reaction. Fluorescence melting curve analysis analyzes the difference in binding affinity between the PCR reaction product and the introduced probe depending on the melting temperature. Unlike other SNP detection probes, the probe is very simple to design, and thus is constructed using an 11 to 18-mer nucleotide sequence including an SNP. Therefore, in order to design a probe having a desired melting temperature, the Tm value may be adjusted according to the length of the PNA probe, and even in the case of PNA probes having the same length, the Tm value may be adjusted by changing the probes. Since PNA has a higher binding affinity than DNA and thus has a higher basic Tm value, PNA may be designed with a shorter length than DNA, and thus can detect even closely neighboring SNPs. In a conventional HRM method, the difference in Tm value is very small at about 0.5° C., and thus an additional analysis program or a minute temperature change are required, and analysis becomes difficult when two or more SNPs appear. However, the PNA probe is not affected by SNPs other than the probe sequence, and thus enables fast and accurate analysis.

The present invention is also directed to a method for detecting multiple chromosomal aneuploidies, which uses at least two primers, at least two assay probes, and at least two elimination probes, and in which the assay probes have different reporters.

It is obvious to those skilled in the art that the method for detecting chromosomal aneuploidy according to the present invention may be applied not only for detection of fetal chromosomal abnormalities, but also for detection of cancer-related chromosomal abnormalities.

In another aspect, the present invention is directed to a PCR composition for detecting chromosomal aneuploidy, the PCR composition comprising:
i) a primer capable of amplifying both a control nucleotide sequence located on a chromosome not associated with chromosomal aneuploidy and a target nucleotide located on a chromosome associated with chromosomal aneuploidy;
ii) an assay probe capable of hybridizing with a sequence that differs by one or two nucleotides from the control nucleotide sequence or the target nucleotide sequence; and
iii) an elimination probe comprising part or all of a sequence of the assay probe, which hybridizes with the target nucleotide sequence or the control nucleotide sequence, the elimination probe having a higher binding affinity than the assay probe.

### EXAMPLES

Hereinafter, the present invention will be described in more detail with reference to examples. It will be obvious to those skilled in the art that these examples serve merely to illustrate the present invention, and the scope of the present invention is not limited to these examples.

### Example 1

### Construction of Primers for Detection of Chromosomal Aneuploidy

For real-time polymerase chain reaction of target nucleotide sequences of chromosomal abnormalities (Down's syndrome (chromosome 21), Edward's syndrome (chromosome 18), and Patau syndrome (chromosome 13)) and internal control nucleotide sequences, primers for Down's syndrome (SEQ ID NOs: 1 to 10), Edward's syndrome (SEQ ID NOs: 11 to 20) and Patau syndrome (SEQ ID NOs: 21 to 30) were constructed (Table 2).

TABLE 2

| SEQ | | | | |
|---|---|---|---|---|
| ID NO | Name | Sequence (5′-3′) | Position (bp)* | Target |
| 1 | DS_F1 | AGAGGTCATAGAAGGTTAT | Chr21: 29,066,953-29,067,073 | Down |
| | | GAAATAGC | Chr1: 147,204,433-147,204,555 | Syndrome |
| 2 | DS_R1 | GAGGTACGAAGTAGAGATG | | |
| | | AGACTTC | | |
| 3 | DS_F2 | CAGCAAGGTTGAAATTGGG | Chr21: 17,517,415-17,517,544 | |
| | | AATG | ChrQ: 52,087,749-52,087,880 | |
| 4 | DS_R2 | GAGTAGGAGAGTGGTTGAG | | |
| | | GAAATCC | | |
| 5 | DS_F3 | CAAACTGGAATAGCTAGCA | Chr21: 17,517,519-17,517,644 | |
| | | TGTGCTTGC | Chr4: 52,087,649-52,087,774 | |
| 6 | DS_R3 | GGACATTCCCAATTTCAAC | | |
| | | CTTGCTG | | |
| 7 | DS_F4 | GGGACATGATTTGTAAAGT | Chr21: 25,769,018-25,769,131 | |
| | | TCAAGGC | Chr7: 63,894,430-63,894,543 | |
| 8 | DS_R4 | CACATTCTGTGACCAAACG | | |
| | | GTTCAAC | | |
| 9 | DS_F5 | CCACAGGGCTAAAGCAACC | Chr21: 33,577,024-33,577,151 | |
| | | ATCTCC | Chr1: 157,711,009-157,711,136 | |
| 10 | DS_R5 | CTCCCTTCTTATGACCCAAG | | |
| | | TGGCT | | |
| 11 | ES_F1 | CAGGGAAAATGACCTTCAC | Chr18: 51,126,794-51,126,903 | Edward |
| | | TGCTG | Chr1: 35,420,274-35,420,383 | Syndrome |
| 12 | ES_R1 | CATCCCCTTTACCTTAGTTT | | |
| | | ACCCAC | | |
| 13 | ES_F2 | GTGCTGGTGGCAGTGTTAT | Chr18: 26,965,287-26,965,406 | |
| | | TTCC | Chr4: 22,614,174-22,614,293 | |
| 14 | ES_R2 | AGTAATGTGTTGTCAGTTC | | |
| | | ACTGAGG | | |
| 15 | ES_F3 | GGAGCTGCGACACGGAGAA | Chr18: 61,816,256-61,816,374 | |
| 16 | ES_R3 | CAAGCACACCTGCTGTTCA | Chr9: 5,418,772-5,418,890 | |
| 17 | ES_F4 | CAGTGCGCGAAATGTAGTT | Chr18: 3,580,328-3,580,443 | |
| | | TTG | Chr15: 84,801,962-84,802,077 | |
| 18 | ES_R4 | GTGTAGCACAAACCACAGA | | |
| | | GGAGAC | | |
| 19 | ES_F5 | CCTCCTCCCTGTCTTCTCTG | Chr18: 3,581,283-3,581,403 | |
| | | ATTC | Chr15: 84,802,918-84,803,038 | |
| 20 | ES_R5 | CAAACAGAGGTGTGCAGCA | | |
| | | GAGG | | |
| 21 | PS_F1 | CTGCCTTTTGAACCAGTTAG | Chr13: 65,442,145-65,442,236 | Patau |
| | | TCTGGAG | Chr3: 12,157,715-12,157,806 | Syndrome |
| 22 | PS_R1 | TCCCTTCTCTACTCTGACTC | | |
| | | CTACC | | |
| 23 | PS_F2 | CCTCAACAGGAGAGCAGAA | Chr13: 21,351,718-21,351,840 | |
| | | GGCTC | Chr6: 5,824,619-5,824,740 | |
| 24 | PS_R2 | GTGTGCTTCAAGGCTCAGT | | |
| | | TAGTG | | |
| 25 | PS_F3 | CCCCATGCTGCCCAGTCCT | Chr13: 21,345,064-21,345,164 | |
| | | G | Chr6: 5,831,357-5,831,457 | |
| 26 | PS_R3 | CTAGGTTCTCTACGGCCTCT | | |
| | | TGTTACT | | |
| 27 | PS_F4 | GCGTTCTTGTTCTCTAGCTT | Chr13: 19,647,040-19,647,128 | |
| | | CCTG | Chr12: 7,438,941-7,439,029 | |
| 28 | PS_R4 | GATACCGATGTCAGAGGCA | | |
| | | GGAGG | | |
| 29 | PS_F5 | GTTTTGTTTCTCTTCTCTGC | Chr13: 19,646,853-19,646,949 | |
| | | TGTCGG | Chr12: 7,439,120-7,439,216 | |
| 30 | PS_R5 | CCAGGTGGAATCTGAATCA | | |
| | | AGTGTAC | | |

### Example 2

### Construction of Fluorescent PNA Probes

For detection of the target nucleotide sequence of chromosomal aneuploidy, bifunctional fluorescent PNA probes (assay probes) having a melting temperature analysis function were constructed. Each of the probes was constructed such that a probe region targeting a sequence region that differs by one or two nucleotides from a control nucleotide sequence having at least 90% homology to a target nucleotide sequence would match the target nucleotide sequence or matches the control nucleotide sequence. A fluorophore (Texas Red) and quencher were attached to the assay probe comprising the target nucleotide sequence (Table 3).

TABLE 3

| SEQ ID | | | | | Perfect |
|---|---|---|---|---|---|
| NO | Name | Sequence(5′-3′) | Fluor | Target | Match |
| 31 | DS_P1 | Dabcyl-ATTTGGTATGTTGTTCTG-O-K | TxR | Down1 | Target |
| 32 | DS_P2 | Dabcyl-TCATCCCCCAACACAA-O-K | TxR | Down2 | Target |
| 33 | DS_P3 | Dabcyl-GTTCTTAATAGCAGGTAC-O- | TxR | Down3 | Target |
| | | K | | | |
| 34 | DS_P4 | Dabcyl-GACTCTTATTGGATACAG-O- | TxR | Down4 | Target |
| | | K | | | |
| 35 | DS_P5 | Dabcyl-GGTATGTTGTGTGATG-O-K | TxR | Down5 | Target |
| 36 | DS_P6 | Dabcyl-GGTATGGTTCCCTTAGA-O-K | TxR | Down6 | Target |
| 37 | DS_P7 | Dabcyl-CCCAGTCGTCAGCAA-O-K | TxR | Down7 | Target |
| 38 | DS_P8 | Dabcyl-CTCACCAAACTCCCAG-O-K | TxR | Down8 | Target |
| 39 | DS_P9 | Dabcyl-GAACCCCGCTAAGG-O-K | TxR | Down9 | Target |
| 40 | DS_P10 | Dabcyl-GGGCTTGTTCAGCT-O-K | TxR | Down10 | Target |
| 41 | ES_P1 | Dabcyl-TTCTGGGTCAAGCCT-O-K | TxR | Edward1 | Target |
| 42 | ES_P2 | Dabcyl-AGCTCCATAGCAGTG-O-K | TxR | Edward2 | Target |
| 43 | ES_P3 | Dabcyl-TGGTCCTCATCTGCTG-O-K | TxR | Edward3 | Target |
| 44 | ES_P4 | Dabcyl-GCTCGAATTTCAGAG-O-K | TxR | Edward4 | Target |
| 45 | ES_P5 | Dabcyl-CACTGGCTTATCATGTCT-O- | TxR | Edward5 | Target |
| | | K | | | |
| 46 | ES_P6 | Dabcyl-ATTATTCCGAACTCTAGC-O- | TxR | Edward6 | Target |
| | | K | | | |
| 47 | ES_P7 | Dabcyl-CAGACCTAAGTTCAAG-O-K | TxR | Edward7 | Target |
| 48 | ES_P8 | Dabcyl-GATGATTCTGAGCACA-O-K | TxR | Edward8 | Target |
| 49 | ES_P9 | Dabcyl-CCCCAGGCTGCTTAT-O-K | TxR | Edward9 | Target |
| 50 | ES_P10 | Dabcyl-TGA CTC TAA AGC AGA-O-K | TxR | Edward10 | Target |
| 51 | PS_P1 | Dabcyl-CTCTAGTTCGCCATAGCC-O- | TxR | Patau1 | Target |
| | | K | | | |
| 52 | PS_P2 | Dabcyl-CCACCATTAGTGCCTCT-O-K | TxR | Patau2 | Target |
| 53 | PS_P3 | Dabcyl-CCTCAAGCCACACAA-O-K | TxR | Patau3 | Target |
| 54 | PS_P4 | Dabcyl-TGTCCTCAGCCTTTCTCG-O-K | TxR | Patau4 | Target |
| 55 | PS_P5 | Dabcyl-CCCTTCACTGTCATCCT-O-K | TxR | Patau5 | Target |
| 56 | PS_P6 | Dabcyl-CCAGCAGCCTCCACA-O-K | TxR | Patau6 | Target |
| 57 | PS_P7 | Dabcyl-GCTGTGTCAGTCCTG-O-K | TxR | Patau7 | Target |
| 58 | PS_P8 | Dabcyl-CAGTTGACATTAGTAAAT-O- | TxR | Patau8 | Target |
| | | K | | | |
| 59 | PS_P9 | Dabcyl-CTCCCGAGCTGACTCC-O-K | TxR | Patau9 | Target |
| 60 | PS_P10 | Dabcyl-AAATCCGCCCTGAC-O-K | TxR | Patau10 | Target |

* In Table 3 above, O- denotes a linker, and K denotes lysine.

### Example 3

### Construction of Elimination Probes

To increase the analytical resolution of the target probe that is used in the detection of chromosomal abnormalities, probes that eliminate both a target nucleotide sequence and a control nucleotide sequence by targeting a sequence region that differs by one or two nucleotides from the control nucleotide sequence having at least 90% homology to the target nucleotide sequence were constructed as set forth in SEQ ID NOs: 61 to 66. In addition, probes (non-fluorescent) that eliminate the target nucleotide sequence were constructed as set forth in SEQ ID NOs: 67 to 71, and probes, which eliminate the target nucleotide sequence and to which a fluorophore and a quencher have been attached, were constructed as set forth in SEQ ID NOs: 72 to 86 (Table 4).

TABLE 4

| SEQ ID | | | | | Perfect |
|---|---|---|---|---|---|
| NO | Name | Sequence (5′-3′) | Fluor | Target | Match |
| 61 | E-Probe 1 | GATACAGTGCAGC | non | Down | Control |
| 62 | E-Probe 2 | GATACAGTGCAGCG | non | Down | Target |
| 63 | E-Probe 3 | GGATACAGTGCAGCG | non | Down | Target |
| 64 | E-Probe 4 | GTGTGATGATCAGC | non | Down | Target |
| 65 | E-Probe 5 | GTGTGATGATCAGCA | non | Down | Control |
| 66 | E-Probe 6 | GTGTGATGATCAGCAC | non | Down | Control |
| 67 | E-Probe 7 | ATTTGGTATGTTGTTCTG | non | Down | Control |
| 68 | E-Probe 8 | TCATCCCCCAACACAA | non | Down | Control |
| 69 | E-Probe 9 | GTTCTTAATAGCAGGTAC | non | Down | Control |
| 70 | E-Probe 10 | GACTCTTATTGGATACAG | non | Down | Control |
| 71 | E-Probe 11 | GGTATGAGGTGTGA | non | Down | Control |
| 72 | E-Probe 12 | Dabcyl-ATTTGGTACGTTGTTCTG- | FAM | Down | Control |
| | | O-K | | | |
| 73 | E-Probe 13 | Dabcyl-TCATCTCCCAGCACAA-O-K | FAM | Down | Control |
| 74 | E-Probe 14 | Dabcyl-GTTCTTAACAGCAGGTAC- | FAM | Down | Control |
| | | O-K | | | |
| 75 | E-Probe 15 | Dabcyl-GACTCTTACTGGATACAG- | FAM | Down | Control |
| | | O-K | | | |
| 76 | E-Probe 16 | Dabcyl-GGTATGAGGTGTGA-O-K | FAM | Down | Control |
| 77 | E-Probe 17 | Dabcyl-TTCTGGATCAAGCCT-O-K | FAM | Edward | Control |
| 78 | E-Probe 18 | Dabcyl-AGCTCCGTAGCAGT-O-K | FAM | Edward | Control |
| 79 | E-Probe 19 | Dabcyl-GGTCCTCGTCTGCTG-O-K | FAM | Edward | Control |
| 80 | E-Probe 20 | Dabcyl-GCTCGAGTTTCAGAG-O-K | FAM | Edward | Control |
| 81 | E-Probe 21 | Dabcyl-CACTGGCTCATCATGTCT- | FAM | Edward | Control |
| | | O-K | | | |
| 82 | E-Probe 22 | Dabcyl-CTCTAGTTCTCCATAGCC- | FAM | Patau | Control |
| | | O-K | | | |
| 83 | E-Probe 23 | Dabcyl-CCACCATCAGTGCCTCT-O- | FAM | Patau | Control |
| | | K | | | |
| 84 | E-Probe 24 | Dabcyl-CCTCAAACCACACAA-O-K | FAM | Patau | Control |
| 85 | E-Probe 25 | Dabcyl-TGTCCTCAACCTTTCTCG-O- | FAM | Patau | Control |
| | | K | | | |
| 86 | E-Probe 26 | Dabcyl-CCCTTCATTGTCATCCT-O-K | FAM | Patau | Control |

* In Table 4 above, O- denotes a linker, and K denotes lysine.

### Example 4

### Verification of PBA Probes Using Standard Cell Lines

For trisomy standard cell lines (Table 5), each primer constructed in Example 1 and each PNA probe constructed in Example 2 were mixed with the DNA extracted from the standard cell line, and then PCR was performed using the CFX96™ Real-Time system (BIO-RAD, USA).

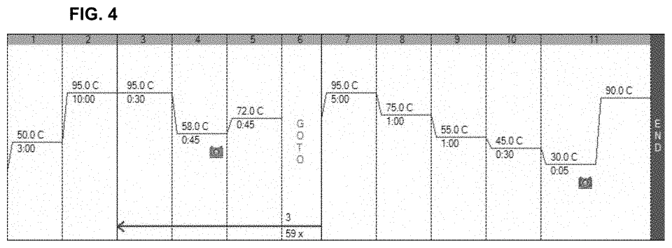

In experimental conditions for real-time polymerase chain reaction, asymmetric PCR was used to produce single-stranded target nucleic acids. Asymmetric PCR was performed under the following conditions: 1 μl of standard cell line DNA (Table 5) was added to 2× SeaSunBio Real-Time FMCA™ buffer (SeaSunBio, Korea), 2.5 mM MgCl₂, 200 μM dNTPs, 1.0 U Taq polymerase, 0.05 μM forward primer (Table 2) and 0.5 μM reverse primer (Table 2) (asymmetric PCR) to reach a total volume of 20 μl, and then real-time PCR was performed, and then 0.5 μl of the fluorescent PNA probe (Table 3) was added thereto and melting curve analysis was performed under the conditions shown in FIG. 4.

TABLE 5

| # | Cell line | |
|---|---|---|
| 1 | NA01137 | Trisomy 21 |
| 2 | NA01920 | Trisomy 21 |
| 3 | NA01921 | Trisomy 21 |
| 4 | NA02067 | Trisomy 21 |
| 5 | NA00143 | Trisomy 18 |
| 6 | NA02422 | Trisomy 18 |
| 7 | NA02732 | Trisomy 18 |
| 8 | NA03623 | Trisomy 18 |
| 9 | NA00526 | Trisomy 13 |
| 10 | NA03330 | Trisomy 13 |
| 11 | NA02948 | Trisomy 13 |
| 12 | NG12070 | Trisomy 13 |

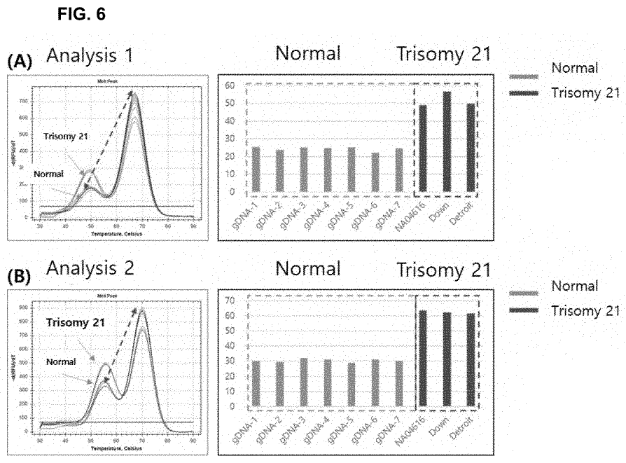

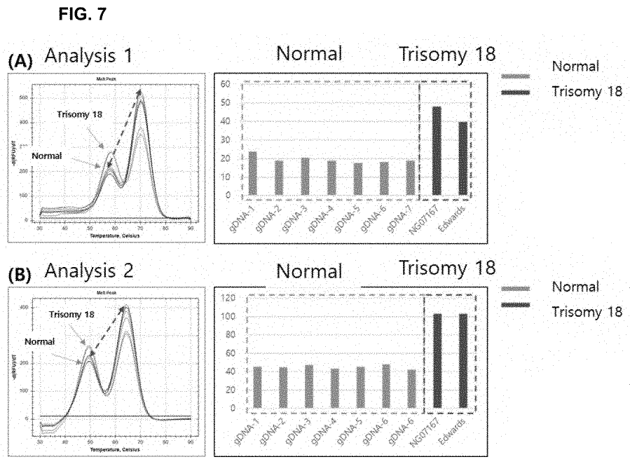

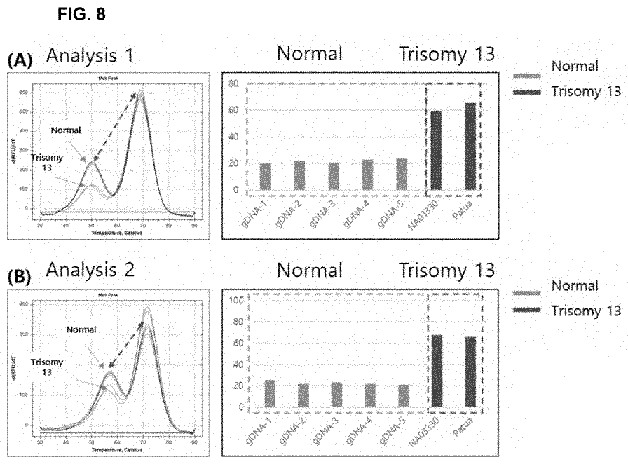

As a result, as shown in FIGS. 6, 7, and 8, it was confirmed that the difference in analysis value (mismatch value/perfect match value) between the trisomic and euploid cell lines appeared.

### Example 5

### Comparative Analysis of Sensitivity of PNA Probe-Based Detection of Down's Syndrome

DNA extracted from the trisomy 21 (Down's syndrome) standard cell line (Table 5) was mixed with euploid normal gDNA at rates of 5, 10, 20, 30 and 100%, and sensitivity was analyzed. The primer and PNA probe constructed in Examples 1 and 2 were added thereto, and then PCR was performed using the CFX96™ Real-Time system (BIO-RAD, USA).

In experimental conditions for real-time polymerase chain reaction, asymmetric PCR was used to produce single-stranded target nucleic acids. Asymmetric PCR was performed under the following conditions: 1 μl of standard cell line DNA (Table 5) was added to 2× SeaSunBio Real-Time FMCA™ buffer (SeaSunBio, Korea), 2.5 mM MgCl₂, 200 μM dNTPs, 1.0 U Taq polymerase, 0.05 μM forward primer (Table 2) and 0.5 μM reverse primer (Table 2) (asymmetric PCR) to reach a total volume of 20 μl, and then real-time PCR was performed, and then 0.5 μl of the fluorescent PNA probe (Table 3) was added thereto and melting curve analysis was performed under the conditions shown in FIG. 4.

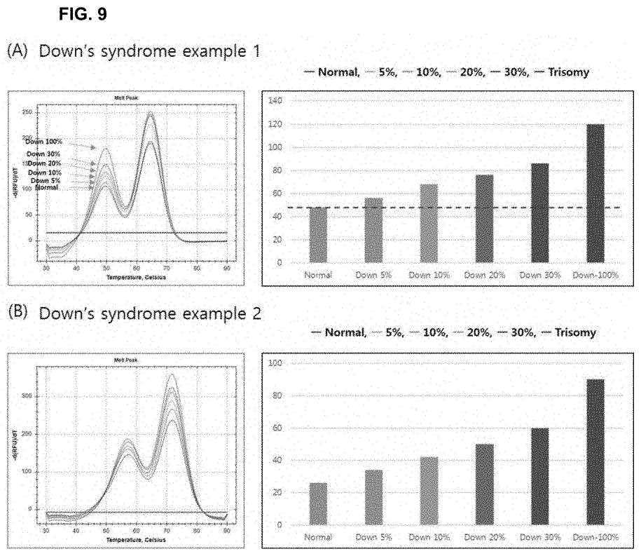

As a result, it was confirmed that analysis of trisomy 21 (Down's syndrome) was possible even in the mixture containing 5% DNA (FIG. 9).

### Example 6

### Verification of Effect of Elimination Probe on Increased Analytical Resolution

To increase analytical resolution for detection of chromosomal abnormality in Examples 4 and 5, PCR was performed in the CFX96™ Real-Time system (BIO-RAD, USA) using the primer, PNA probe and non-fluorescent elimination probe constructed in Examples 1, 2 and 3.

In experimental conditions for real-time polymerase chain reaction, asymmetric PCR was used to produce single-stranded target nucleic acids. The asymmetric PCR was performed under the following conditions: 1 μl of standard cell line DNA (Table 5) was added to 2× SeaSunBio Real-Time FMCA™ buffer (SeaSunBio, Korea), 2.5 mM MgCl₂, 200 μM dNTPs, 1.0 U Taq polymerase, 0.05 μM forward primer (Table 2) and 0.5 μM reverse primer (Table 2) (asymmetric PCR) to reach a total volume of 20 μl, and then real-time PCR was performed. Then, 0.5 μl fluorescent PNA probe (Table 3) and each non-fluorescent elimination probe (Table 4, E-Probes 1 to 11) were added thereto and melting curve analysis was performed under the conditions shown in FIG. 4.

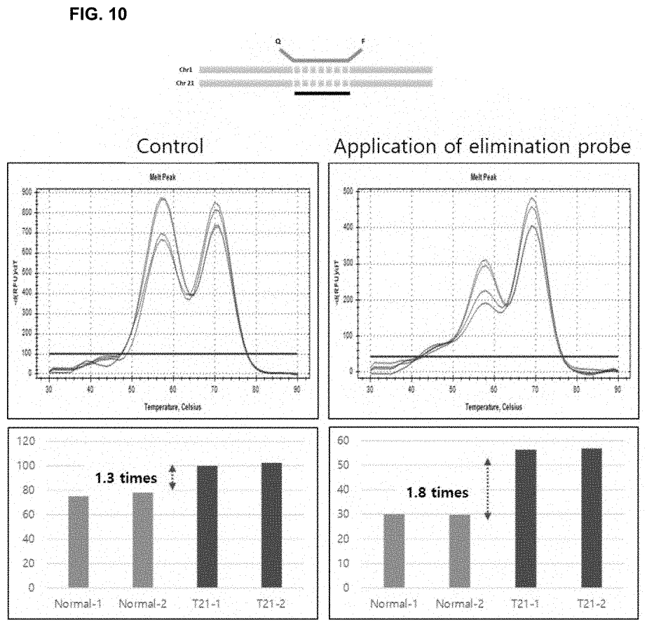

The case where the assay probe was used alone was compared with the case where the assay probe and the elimination probe were used in combination. It was confirmed that the resolution was higher when the non-fluorescent probe that eliminates only the control sequence was used (the difference between normal and abnormal was 1.8 times) than when the conventional assay probe was used alone (the difference between normal and abnormal was 1.3 times; FIG. 10).

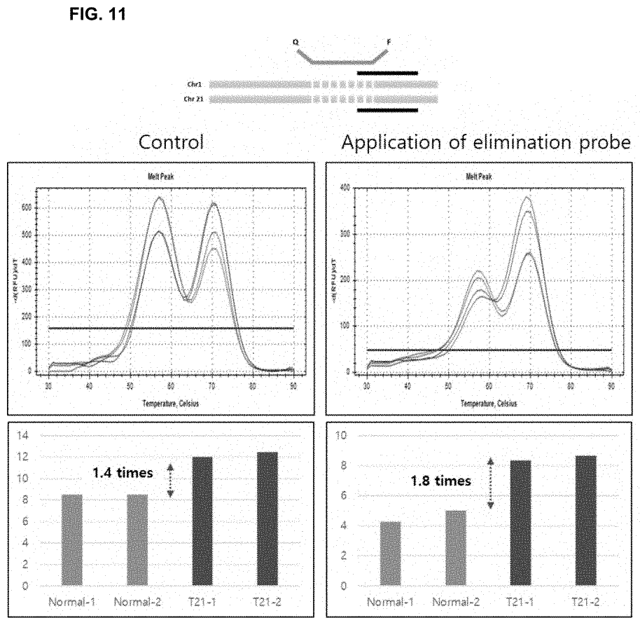

In addition, it was confirmed that, even when the non-fluorescent probe that eliminates the target nucleotide sequence and the control sequence was used, the difference between normal and abnormal was 1.8 times, and the resolution was higher than that in the conventional analysis method (the difference between normal and abnormal was 1.4 times) (FIG. 11).

### Example 7

### Verification of Effect of Result Correction on Increased Analytical Resolution

To increase analytical resolution for detection of chromosomal abnormality in Examples 4 and 5, PCR was performed in the CFX96™ Real-Time system (BIO-RAD, USA) using the primer, PNA probe and non-fluorescent elimination probe constructed in Examples 1, 2 and 3.

In experimental conditions for real-time polymerase chain reaction, asymmetric PCR was used to produce single-stranded target nucleic acids. Asymmetric PCR was performed under the following conditions: 1 μl of standard cell line DNA (Table 5) was added to 2× SeaSunBio Real-Time FMCA™ buffer (SeaSunBio, Korea), 2.5 mM MgCl₂, 200 μM dNTPs, 1.0 U Taq polymerase, 0.05 μM forward primer (Table 2) and 0.5 μM reverse primer (Table 2) (asymmetric PCR) to reach a total volume of 20 μl, and then real-time PCR was performed. Then, 0.5 μl fluorescent PNA probe (Table 3) and each fluorescent elimination probe (Table 4, E-Probes 12 to 261) were added thereto and melting curve analysis was performed under the conditions shown in FIG. 4.

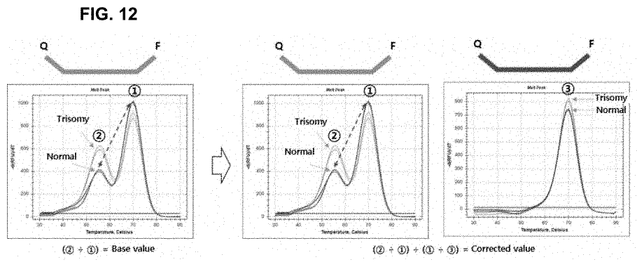

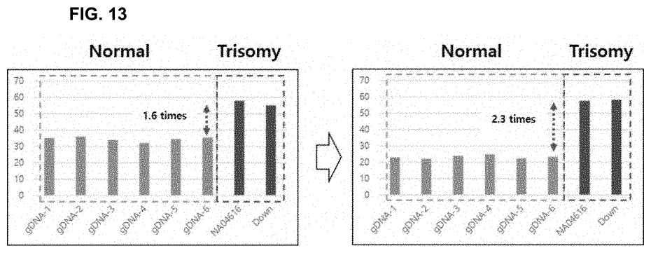

Correction of the results was performed using the fluorescent elimination probe targeting the control nucleotide sequence (FIG. 12). It was confirmed that the analytical resolution after result correction (mismatch value/perfect match value)/(perfect match value/perfect match value by elimination probe) increased compared to that before correction (mismatch value/perfect match value) (1.6 times→2.3 times; FIG. 13).

### Example 8

Verification of Down's Syndrome Detection using Clinical Sample

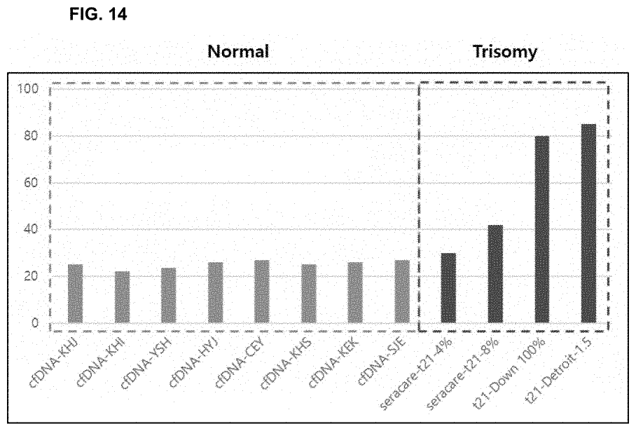

cfDNA extracted from normal maternal blood was analyzed comparatively with a trisomy 21 standard substance (trisomy 21, Down's syndrome). As the trisomy 21 standard substance (trisomy 21, Down's syndrome), Seraseg™ Trisomy 21 Aneuploidy Linearity Panel (4-8% Fetal Fraction) and a standard cell line were used. As a result, as shown in FIG. 14, it was confirmed that the results obtained for 4% and 8% trisomy 21 standard substances (trisomy 21, Down's syndrome) were all different from the results obtained for the normal maternal cfDNA, indicating that chromosomal abnormalities could be detected (FIG. 14).

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

### INDUSTRIAL APPLICABILITY

The method for detecting chromosomal aneuploidy according to the present invention may analyze the ratio of the target nucleotide sequence to the control nucleotide sequence at high resolution by eliminating equal amounts (certain proportions) of the target nucleotide sequence and the control nucleotide sequence from the analysis using the elimination sequence. This method is useful because numerical abnormalities (aneuploidy) in chromosomes (e.g., fetal chromosomes in maternal blood, and circulating tumor DNA in cancer patients) present at low rates can be detected quickly with high sensitivity by the use of this method.

1. A method for detecting chromosomal aneuploidy, the method comprising steps of:
a) isolating DNAs from a normal sample and a sample derived from a patient expected to have chromosomal aneuploidy, respectively;
b) performing amplification using a primer capable of amplifying both a control nucleotide sequence located on a chromosome not associated with chromosomal aneuploidy and a target nucleotide located on a chromosome associated with chromosomal aneuploidy;
c) hybridizing the amplification products with an assay probe capable of hybridizing to a sequence, which differs by one or two nucleotides from the control nucleotide sequence or the target nucleotide sequence, and with an elimination probe comprising part or all of a sequence of the assay probe, which hybridizes with the target nucleotide sequence or the control nucleotide sequence, the elimination probe having a higher binding affinity for the amplification products of step b) than the assay probe; and
d) identifying chromosomal aneuploidy by analyzing melting curves of the hybridization products for the normal sample and the subject sample, obtained in step c).

2. The method of claim 1, wherein a primer or probe hybridization region of the control nucleotide sequence of step b) is at least 90% homologous to a primer or probe hybridization region of the target nucleotide sequence.

3. The method of claim 1, wherein the assay probe of step c) has a melting temperature difference of 8° C. or more when it either perfectly matches or mismatches the control nucleotide sequence or the target nucleotide sequence.

4. The method of claim 1, wherein the assay probe of step c) is a peptide nucleic acid (PNA), and a reporter and a quencher are attached to both ends of the assay probe.

5. The method of claim 4, wherein the reporter is at least one selected from the group consisting of FAM (6-carboxyfluorescein), Texas red, HEX (2′,4′,5′,7′-tetrachloro-6-carboxy-4,7-dichlorofluorescein) and Cy5.

6. The method of claim 4, wherein the quencher is at least one selected from the group consisting of TAMRA (6-carboxytetramethyl-rhodamine), BHQ1, BHQ2 and Dabcyl.

7. The method of claim 1, wherein the elimination probe of step c) is selected from the group consisting of: a probe for eliminating only the product of amplification of the target nucleotide sequence; and a probe for eliminating both the products of amplification of the target nucleotide sequence and the control nucleotide sequence.

8. The method of claim 7, wherein the elimination probe of step c) hybridizes with the product of amplification of the control nucleotide sequence or the target nucleotide sequence competitively with the assay probe.

9. The method of claim 8, wherein the elimination probe is selected from the group consisting of an oligonucleotide, LNA, PNA, and combinations thereof.

10. The method of claim 1, wherein the elimination probe of step c) eliminates 50 to 90% of the amplification products of step b).

11. The method of claim 1, wherein analysis of the melting curves in step d) is performed by a method comprising steps of:
a) calculating the mismatch value/perfect match value ratio of the product of amplification of the normal sample DNA;
b) calculating the mismatch value/perfect match value ratio of the product of amplification of the subject sample DNA; and
c) determining that the subject sample is normal when the ratio calculated in step a) is the same as the ratio calculated in step b), and has chromosomal aneuploidy when the ratio calculated in step a) is different from the ratio calculated in step b).

12. The method of claim 11, wherein the method for performing analysis of the melting curves further comprises: step d) of correcting the perfect match value, obtained by the elimination probe, using the following Equation 1 when calculating the ratios in step a) and step b):
$$Equation 1$$
$$\frac{Mismatch value}{Perfect match value} / \frac{Perfect match value}{Perfect match value by elimination probe}$$

13. The method of claim 1, which is a method for detecting multiple chromosomal aneuploidies, which uses at least two primers, at least two assay probes, and at least two elimination probes, in which the assay probes have different reporters.

14. A PCR composition for detecting chromosomal aneuploidy, the PCR composition comprising:
i) a primer capable of amplifying both a control nucleotide sequence located on a chromosome not associated with chromosomal aneuploidy and a target nucleotide located on a chromosome associated with chromosomal aneuploidy;
ii) an assay probe capable of hybridizing with a sequence that differs by one or two nucleotides from the control nucleotide sequence or the target nucleotide sequence; and
iii) an elimination probe comprising part or all of a sequence of the assay probe, which hybridizes with the target nucleotide sequence or the control nucleotide sequence, the elimination probe having a higher binding affinity than the assay probe.

15. The method of claim 1, wherein the primer is selected from a group consisting of SEQ ID NOS:1 to 30.

16. The method of claim 1, wherein the assay probe is selected from a group consisting of SEQ ID NOS:31 to 60.

17. The method of claim 1, wherein the elimination probe is selected from a group consisting of SEQ ID NOS:61 to 86.
